(19) United States
(12) Patent Application Publication
(10) Pub. No.: US 2015/0299815 A1
(43) Pub. Date: Oct. 22, 2015

(54) ORGANIC MATTER PRODUCTION METHOD, ORGANIC MATTER PRODUCTION PROCESS MONITORING METHOD, AND ORGANIC MATTER PRODUCTION PROCESS MONITORING DEVICE

(71) Applicant: SUMITOMO ELECTRIC INDUSTRIES, LTD., Osaka-shi, Osaka (JP)

(72) Inventors: Tetsu MORISHIMA, Yokohama-shi (JP); Hiroshi SUGANUMA, Yokohama-shi (JP); Masumi ITOU, Yokohama-shi (JP); Miyoko FUJIMOTO, Yokohama-shi (JP); Akinori KIMURA, Yokohama-shi (JP); Yoko IGARASHI, Yokohama-shi (JP)

(21) Appl. No.: 14/654,147

(22) PCT Filed: Dec. 11, 2013
(86) PCT No.: PCT/JP2013/083172

§ 371 Date: Jun. 19, 2015

(30) Foreign Application Priority Data
Dec. 25, 2012 (JP) 2012-281300

(51) Int. Cl. C12Q 3/00; C12Q 1/02; G01N 33/50; C12P 7/06

(52) U.S. Cl. CPC C12Q 3/00; C12P 7/06; C12Q 1/02; G01N 33/5005

(57) ABSTRACT

An organic matter production method according to the present invention in which production process status can be more properly monitored includes a first step of acquiring absorbance spectrum of as measurement object in which the amount of the raw material or desired product varies with progress of a production process by receiving transmitted light or diffuse reflected light from the measurement object as a result of broadband light irradiation of the measurement object, a second step of extracting two or more feature values indicative of the characteristics of the measurement object from the absorbance spectrum, and a third step of controlling the production process on the basis of the two or more feature values.

### TECHNICAL FIELD

The present invention relates to an organic matter production method, a process monitoring method for organic matter, and a process monitor for organic matter.

### BACKGROUND ART

As a process monitoring method in the production of a product containing organic matter by fermentation or cell culture, Japanese Unexamined Patent Application Publication No. 5-273124 describes a method for measuring the acidity of fermented milk by irradiating the fermented milk with broadband light having a wavelength in the range of 700 to 1200 nm. International Publication No. WO 2007/052716 describes an apparatus that acquires a cell image with a CCD camera, determines the culture state from the image, and performs culture operation. Japanese Unexamined Patent Application Publication No. 2008-76409 describes a method for determining transplantability from hardness or elasticity information of a cultured tissue measured with an oscillator. Japanese Unexamined Patent Application Publication No. 2010-81823 describes an apparatus that controls the culture state on the basis of the cell size determined from a cell image taken with a camera. The production process status of a measurement object could not be sufficiently recognized by using these methods of the related art.

### SUMMARY OF INVENTION

### Technical Problem

It is an object of the present invention to provide an organic matter production method, a process monitoring method for organic matter, and a process monitor for organic matter that can more properly recognize the production process status.

### Solution to Problem

To this end, there is provided a method for producing a desired product containing organic matter from a raw material that includes (1) a first step of acquiring an absorbance spectrum of a measurement object in which the amount of the raw material or desired product varies with progress of a production process by receiving transmitted light or diffuse reflected light from the measurement object as a result of broadband light irradiation of the measurement object, (2) a second step of extracting two or more feature values indicative of the characteristics of the measurement object from the absorbance spectrum, and (3) a third step of controlling the production process on the basis of the two or more feature values.

In an organic matter production method according to the present invention, the production process may be microorganism fermentation, culture of animal cells, plant cells, or a microorganism, or a chemical reaction. In the first step, the absorbance spectrum of the measurement object may be intermittently acquired multiple times. In the second step, a change in the two or more feature values indicative of the characteristics of the measurement object may be determined from the absorbance spectra acquired in the first step. In the third step, the production process may be controlled on the basis of the change in the feature values determined in the second step. In the second step, the feature values may be extracted from a second derivative of the absorbance spectrum, or the feature values may be extracted through a multivariate analysis of the absorbance spectrum. The broadband light preferably includes light having a wavelength in the range of 1000 to 2500 nm.

Another aspect of the present invention provides a process monitoring method for organic matter that monitors the progress of a production process in the production of a desired product containing organic matter from a raw material. The process monitoring method includes: (1) a first step of acquiring an absorbance spectrum of a measurement object in which the amount of the raw material or desired product varies with progress of the production process by receiving transmitted light or diffuse reflected light from the measurement object as a result of broadband light irradiation of the measurement object, and (2) a second step of extracting two or more feature values indicative of the characteristics of the measurement object from the absorbance spectrum.

Still another aspect of the present invention provides a process monitor for organic matter that monitors the progress of a production process in the production of a desired product containing organic matter from a raw material. The process monitor includes a light source unit for emitting broadband light to a measurement object in which the amount of the raw material or desired product varies with progress of the production process, an acquisition unit for acquiring an absorbance spectrum of the measurement object by receiving transmitted light or diffuse reflected light from the measurement object as a result of light irradiation from the light source unit, and an analyzing unit for extracting two or more feature values indicative of the characteristics of the measurement object from the absorbance spectrum acquired by the acquisition unit.

### Advantageous Effects of Invention

In accordance with the present invention, two or more feature values indicative of the characteristics of the measurement object are extracted from the absorbance spectrum of the measurement object resulting from broadband light irradiation. The production process status can be more precisely recognized from the feature values. The organic matter can be more efficiently produced by performing production control, such as parameter management, on the basis of the precisely recognized production process status.

### BRIEF DESCRIPTION OF DRAWINGS

FIG. 1 is a schematic view of a process monitor for organic matter according to an embodiment of the present invention.

FIG. 2 is a graph of second derivatives of absorbance spectra measured with a process monitor for organic matter according to the present invention.

FIG. 3 is a graph showing a correlation between the total sugar concentration and the minimum second derivative of an absorbance spectrum at a wavelength of approximately 1200 nm.

FIG. 4 is a graph showing a correlation between the ethanol concentration and the minimum second derivative of an absorbance spectrum at a wavelength of approximately 1700 nm.

FIG. 5 is a graph of the concentrations of ethanol and sugar in a fermented liquid as a function of time.

FIG. 6 is a graph of second derivatives of absorbance spectra for samples in which the ratio of glucose solution to culture medium is different.

### DESCRIPTION OF EMBODIMENTS

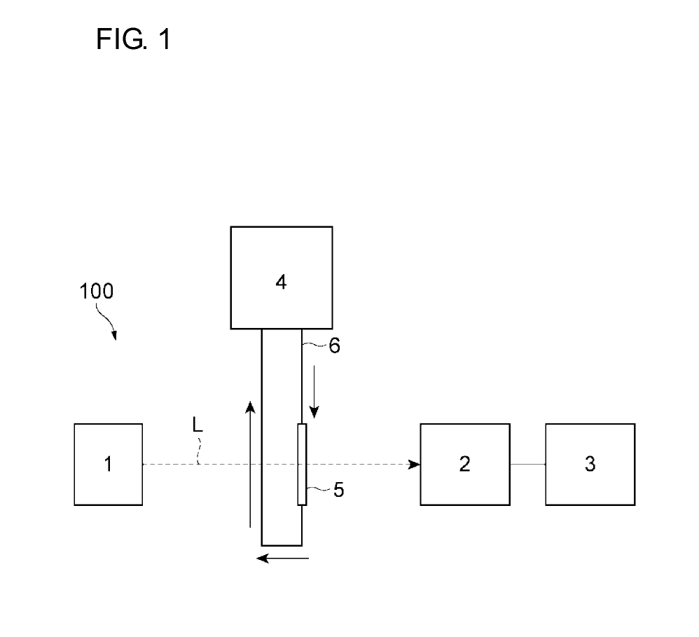

Embodiments of the present invention will be described in detail below with reference to the accompanying drawings. FIG. 1 is a schematic view of a process monitor 100 for organic matter according to an embodiment of the present invention. The process monitor 100 for organic matter includes a light source unit 1, a NIR spectral sensor 2 (an acquisition unit), and an analyzing unit 3. A tank 4 is part of an apparatus for producing a desired product from a raw material. A cell 5 and a tube 6 are mechanisms attached to the tank 4 in order to transfer a measurement object from the tank 4 for measurement with the process monitor 100 for organic matter.

The process monitor 100 for organic matter is used to monitor the progress of a production process in an organic matter production method by which a desired product can be produced from a raw material in the production process. The desired product is organic matter. The production process may be microorganism fermentation, culture of animal cells, plant cells, or a microorganism (bacterium or yeast), or a chemical reaction.

The process monitor 100 for organic matter has a function of monitoring the progress of a production process by assessing a component of a measurement object in which the amount of raw material or desired product varies with the progress of the production process. In the case that the production process is microorganism fermentation, with the progress of the production process, a raw material carbohydrate is decomposed into a desired product (for example, an alcohol). Thus, the measurement object is a mixture of the raw material and the desired product. In the case that the production process is culture of animal cells, plant cells, or a microorganism (bacterium or yeast), with the progress of the production process, the cells or microorganism consumes a nutrient in a culture medium. Thus, the measurement object is the culture medium containing the nutrient. In the case that the production process is a chemical reaction, with the progress of the production process, an unreacted substance decreases, and a reaction product increases. Thus, the measurement object is the unreacted substance and the reaction product.

The light source unit 1 emits broadband light. Light emitted from the light source unit 1 includes light having a wavelength band in the range of 1000 to 2500 nm. Light L emitted from the light source unit 1 is transmitted through an optically transparent cell 5 containing a measurement object and is received by the NIR spectral sensor 2. In the NIR spectral sensor 2, the light L from the measurement object is separated into its spectral components, and the transmitted light intensity for each wavelength is measured to acquire an absorbance spectrum or transmission spectrum. Information about the absorbance spectrum or transmission spectrum is transmitted from the NIR spectral sensor 2 to the analyzing unit 3. Although light transmitted through the measurement object is received by the NIR spectral sensor 2 in the present embodiment, reflected light from the measurement object may be received to acquire a reflection spectrum.

In the tank 4, a production process, that is, fermentation, culture, or a chemical reaction occurs, and a raw material and a desired product are mixed. Part of the contents of the tank 4 are transferred to the cell 5 through the tube 6 and are returned from the cell 5 to the tank 4. This structure may be modified in a manner that depends on the production process to be monitored with the process monitor 100 for organic matter.

The analyzing unit 3 analyzes the absorbance spectrum transmitted from the NIR spectral sensor 2 and extracts two or more feature values indicative of the characteristics of the measurement object. The feature values to be extracted include the amount of component(s) that allows monitoring of the progress of the production process from the raw material to the desired product, such as the ratio of the raw material to the desired product, the amount of reaction inhibitor, and pH. The feature values may be extracted by a method of utilizing a second derivative of the absorbance spectrum, by a standard normal variate transformation method of the absorbance spectrum, or by a multivariate analysis method of the absorbance spectrum. The absorbance spectrum may be subjected to statistical treatment in order to extract the feature values.

The feature values thus extracted from the absorbance spectrum may be used to estimate the concentration of a measurement object component in the mixture on the basis of a predetermined relationship between the concentration of the measurement object component and the feature values of the absorbance spectrum. The feature values may also be used to determine whether the production process reaches a predetermined level by judging whether the feature values exceed a predetermined threshold. Alternatively, the feature values may be extracted from each of absorbance spectra intermittently acquired with the process monitor 100 for organic matter, and the progress of the production process can be monitored from variations in the feature values over time.

The feature values obtained with the process monitor 100 for organic matter may be used to control the production process. An organic matter production method for producing organic matter with the process monitor 100 for organic matter includes a first step of acquiring an absorbance spectrum of a measurement object by receiving transmitted light from the measurement object by the NIR spectral sensor 2 as a result of irradiation of the measurement object with broadband light emitted from the light source unit 1, a second step of extracting two or more feature values from the absorbance spectrum acquired in the first step in the analyzing unit 3, and a third step of controlling the production process on the basis of the feature values extracted in the second step. In the third step, for example, the feature values may be used to control the temperature or humidity of the tank 4. Thus, the desired product can be more efficiently produced in the production process by controlling the production process on the basis of the feature values.

A process monitoring method for organic matter using the process monitor 100 for organic matter will be described below with examples.

### Example 1

### Application to Microorganism Fermentation Process

The total sugar concentration and the ethanol concentration of a measurement object that simulated a bioethanol fermented liquid were measured as feature values using the process monitor 100 for organic matter. In this example, the measurement object was a mixture of sugar (glucose+xylose), ethanol, and water. While the total sugar concentration and the ethanol concentration were maintained at 20% by weight in total, their ratio was changed (from total sugar concentration 20% by weight+ethanol concentration 0% by weight to total sugar concentration 0% by weight+ethanol concentration 20% by weight). An absorbance spectrum at a near-infrared wavelength in the range of 1150 to 1750 nm was acquired for each measurement object using the process monitor 100 for organic matter.

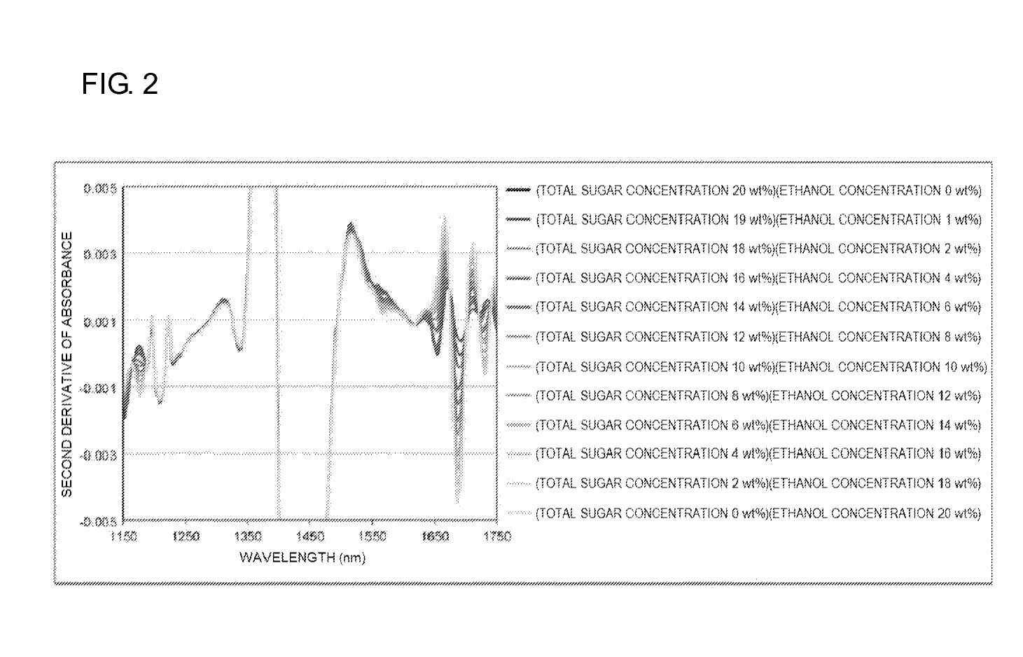

FIG. 2 is a graph of second derivatives of absorbance spectra thus measured. Peaks at wavelengths of approximately 1200 and 1700 nm (the minimum second derivatives of absorbance) varied with changes in the total sugar concentration and the ethanol concentration.

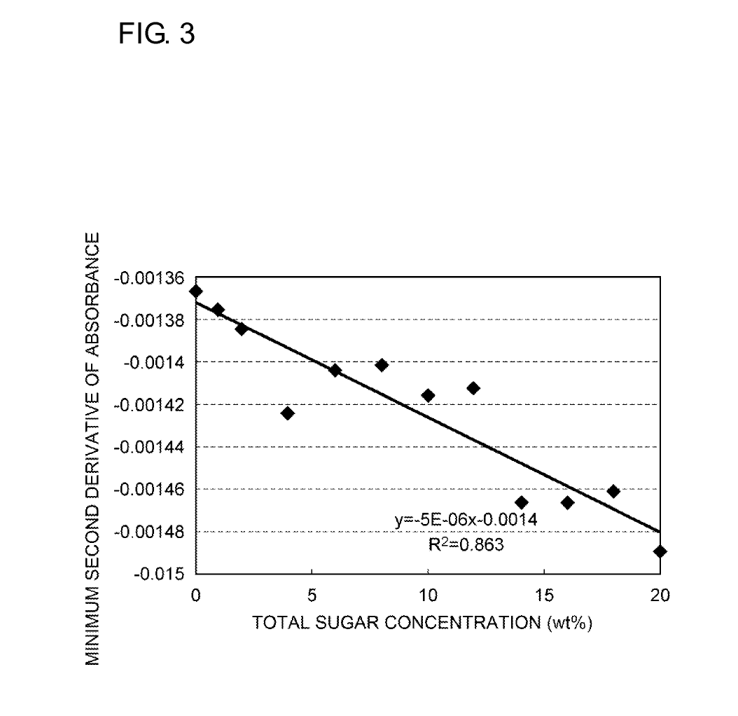

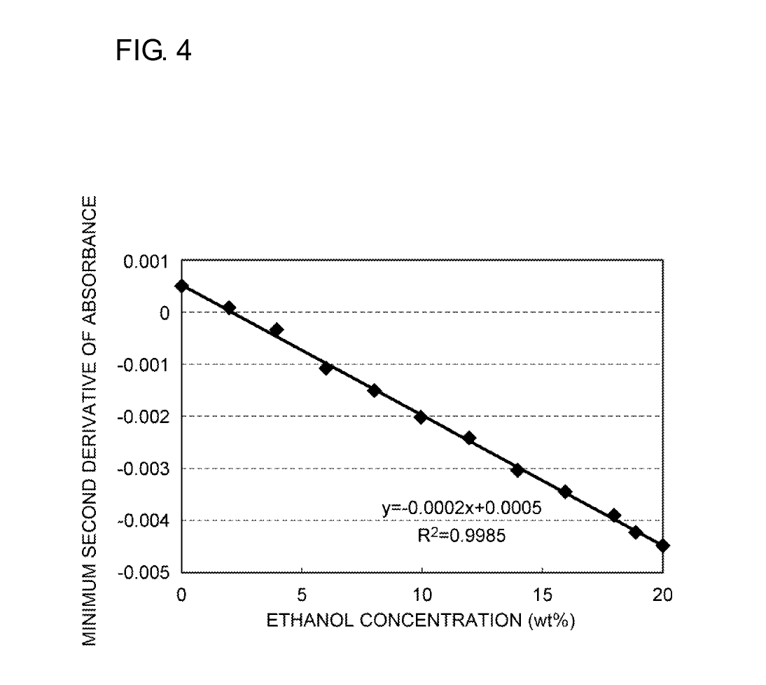

FIG. 3 is a graph showing a correlation between the total sugar concentration and the minimum second derivative at a wavelength of approximately 1200 nm. FIG. 4 is a graph showing a correlation between the ethanol concentration and the minimum second derivative at a wavelength of approximately 1700 nm. These peaks having the wavelengths of the minimum second derivatives are assigned to sugar and ethanol. Thus, the second derivatives of absorbance at a wavelength band including approximately 1200 and 1700 nm can be used to simultaneously measure the two feature values of the total sugar concentration and the ethanol concentration of the aqueous bioethanol.

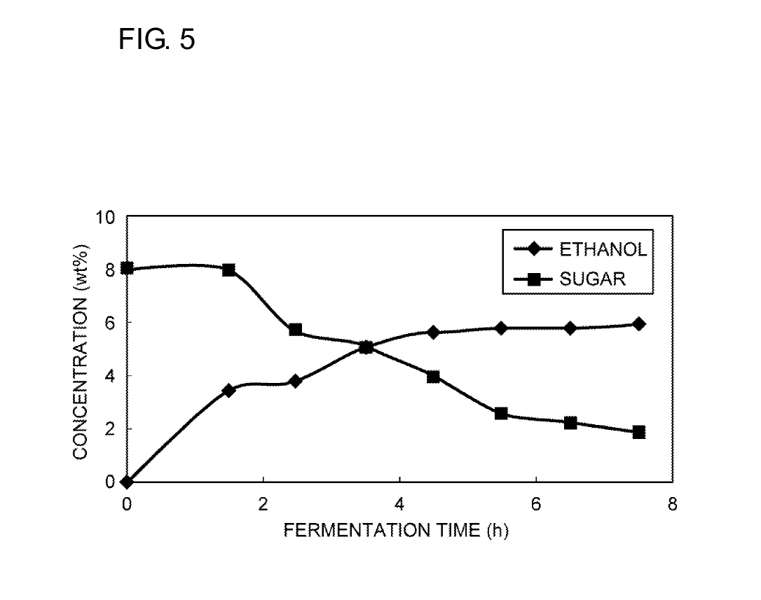

The concentrations of the raw material and the desired product in the fermented liquid can be measured in real time by intermittently acquiring the absorbance spectrum of the measurement object during the progress of the production process, thereby allowing bioethanol fermentation process parameters to be controlled. In the bioethanol fermentation, process parameters, such as fermentation temperature and humidity, can be controlled with reference to the measured sugar and ethanol concentrations, thereby achieving an efficient fermentation environment. FIG. 5 is a graph of the concentrations of ethanol and sugar in the fermented liquid as a function of time. The concentration of a component in the bioethanol fermented liquid is measured at predetermined intervals. Observation of changes in the concentration allows the fermentation process to be controlled according to the progress of the fermentation process.

### Example 2

### Application to Culture Process

Control of variations in the amount of glucose in a culture medium for use in animal cell culture will be described below. In an organic matter production process utilizing animal cells, plant cells, or a microorganism, a desired substance is generally collected after a certain period of culture. In this production process, the yield of a target substance depends on the control of an appropriate amount of nutrient in the culture medium serving as a nutrient source for the cells or microorganism. For example, animal cells generally consume sugar as an energy source, and culture of animal cells decreases the sugar content of the culture medium. Thus, the sugar content of the culture medium must be properly controlled.

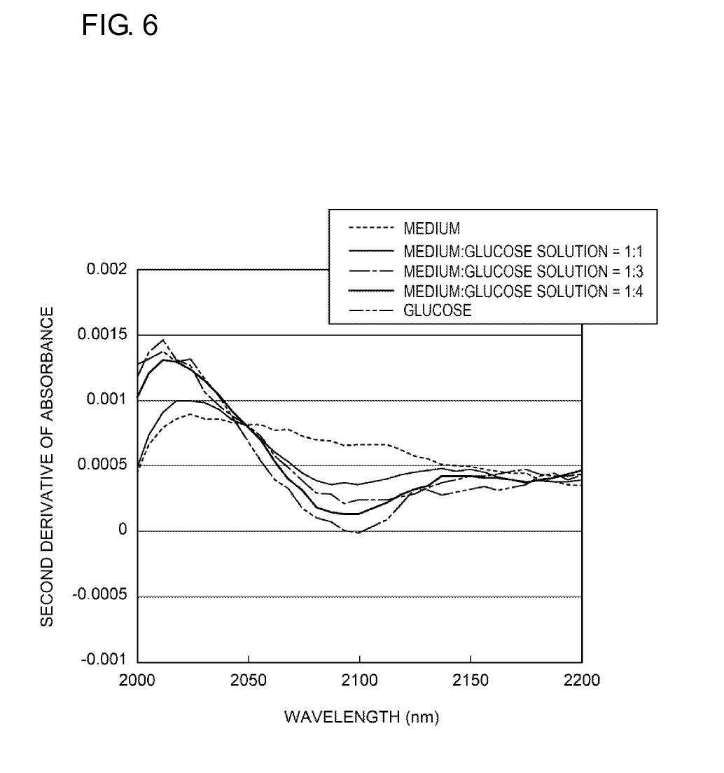

A predetermined amount of glucose solution was added to a culture medium to prepare a measurement object. The absorbance spectrum was measured with the process monitor 100 for organic matter. Five samples were prepared: mixtures of the culture medium and the glucose solution (culture medium:glucose solution=1:1, 1:3, and 1:4), the culture medium alone, and the glucose solution alone. The absorbance spectra of the five samples were measured. FIG. 6 shows the results (second derivatives of the absorbance spectra).

Peak values of the spectra at a wavelength of approximately 2100 nm varied with the amount of added glucose solution (the concentration of the glucose solution in the culture medium). This peak wavelength is characteristic of glucose. Thus, the culture process can be controlled by correlating the peak values at a wavelength of approximately 2100 nm with the concentration of glucose in the culture medium. Likewise, the correlation between the concentration of a component or a product produced by cells in another culture medium and the corresponding spectrum can be used to apply the production process monitoring to various raw materials and desired products. Although one feature value (glucose concentration) has been described above, another feature value (for example, the concentration of a product produced by cells, which varies with cell number) can be simultaneously measured to improve evaluation.

### Example 3

### Application to Chemical Reaction Process

Application to a poly(lactic acid) production process will be described below. Poly(lactic acid) is produced by dehydration condensation of the raw material lactic acid, for example, caused by heating. Poly(lactic acid) formed by condensation can be characterized by parameters such as OH value, water content, and degree of crystallinity. These parameters can be simultaneously determined from an absorbance spectrum, which is measured by irradiating poly(lactic acid) with broadband light.

By way of example, the OH value and the water content are simultaneously determined as described below. The OH value can be correlated with the vibration peak value of the OH group in the lactic acid structure, and the water content can be correlated with the vibration peak value of the OH group of water. In the light wavelength band used in a process monitoring method for organic matter according to the present embodiment, the vibration peak of the OH group in the lactic acid structure is differentiated from the vibration peak of the OH group of water. Thus, these peaks can be used to extract feature values, and thereby the OH value and the water content can be simultaneously and individually determined.

In the present embodiment, an organic matter production process is monitored utilizing an absorbance spectrum, which is measured by irradiating a measurement object with broadband light. Thus, feature values can be obtained in real time and in a noncontact and noninvasive manner in the condensation environment. Feature values obtained by the process monitoring method for organic matter can be used for process control, such as optimization of heating conditions in a condensation reaction.

Although the embodiments of the present invention have been described, the present invention should not be limited to these embodiments, and various modifications may be made therein. For example, although two feature values were assessed in the embodiments, three or more feature values may be assessed. Three or more feature values will allow the production process status to be recognized with a higher degree of precision.

### INDUSTRIAL APPLICABILITY

The present invention can be applied to an organic matter production method utilizing microorganism fermentation, culture of animal cells, plant cells, or a microorganism, or a chemical reaction, a process monitoring method for use in the organic matter production method, and a process monitoring apparatus.

1. A method for producing a desired product containing organic matter from a raw material, comprising:
a first step of acquiring an absorbance spectrum of a measurement object in which the amount of the raw material or desired product varies with progress of a production process by receiving transmitted light or diffuse reflected light from the measurement object as a result of broadband light irradiation of the measurement object;
a second step of extracting two or more feature values indicative of the characteristics of the measurement object from the absorbance spectrum; and
a third step of controlling the production process on the basis of the two or more feature values.

2. The method for producing organic matter the desired product according to claim 1, wherein the production process is microorganism fermentation.

3. The method for producing organic matter the desired product according to claim 1, wherein the production process is culture of animal cells, plant cells, or a microorganism.

4. The method for producing organic matter the desired product according to claim 1, wherein the production process is a chemical reaction.

5. The method for producing organic matter the desired product according to claim 1, wherein
the absorbance spectrum of the measurement object is intermittently acquired multiple times in the first step,
a change in the two or more feature values indicative of the characteristics of the measurement object is determined from the absorbance spectra in the second step, and the production process is controlled on the basis of the change in the two or more feature values in the third step.

6. The method for producing organic matter the desired product according to claim 1, wherein the feature values are extracted from a second derivative of the absorbance spectrum in the second step.

7. The method for producing organic matter the desired product according to claim 1, wherein the feature values are extracted through a multivariate analysis of the absorbance spectrum in the second step.

8. The method for producing organic matter the desired product according to claim 1, wherein the broadband light includes light having a wavelength in the range of 1000 to 2500 nm.

9. A process monitoring method for organic matter that monitors progress of a production process in production of a desired product containing organic matter from a raw material, comprising:
a first step of acquiring an absorbance spectrum of a measurement object in which the amount of the raw material or desired product varies with progress of the production process by receiving transmitted light or diffuse reflected light from the measurement object as a result of broadband light irradiation of the measurement object; and
a second step of extracting two or more feature values indicative of the characteristics of the measurement object from the absorbance spectrum.

10. A process monitor for organic matter that monitors progress of a production process in production of a desired product containing organic matter from a raw material, comprising:
a light source unit for emitting broadband light to a measurement object in which the amount of the raw material or desired product varies with progress of the production process;
an acquisition unit for acquiring an absorbance spectrum of the measurement object by receiving transmitted light or diffuse reflected light from the measurement object as a result of light irradiation from the light source unit; and
an analyzing unit for extracting two or more feature values indicative of the characteristics of the measurement object from the absorbance spectrum.
